(19) United States
(12) Patent Application Publication
(10) Pub. No.: US 2023/0347355 A1
(43) Pub. Date: Nov. 2, 2023

(54) Multilayer Adhesive Fluid Collection Articles Including Capillary Channels

(71) Applicant: 3M INNOVATIVE PROPERTIES COMPANY, St. Paul, MN (US)

(72) Inventors: Steven P. Swanson, Blaine, MN (US); Kurt J. Halverson, Lake Elmo, MN (US); Alexander C. Eldredge, Mahtomedi, MN (US)

(21) Appl. No.: 17/778,886

(22) PCT Filed: Dec. 16, 2020
(86) PCT No.: PCT/IB2020/062052

§ 371 Date: May 23, 2022

Related U.S. Application Data
(60) Provisional application No. 62/951,080, filed on Dec. 20, 2019.

(51) Int. Cl. B01L 9/00; A61B 5/00; B01L 3/00

(52) U.S. Cl. CPC B01L 9/527; A61B 5/4266; B01L 3/5027; B01L 2300/0636; B01L 2400/0406

(57) ABSTRACT

The present disclosure provides articles including an adhesive layer; a film layer bonded to the adhesive layer having a microstructured surface including numerous capillary channels; and a cover layer disposed on the capillary channels and attached to the adhesive layer. The adhesive layer is hydrophobic and includes an aperture for fluid (e.g., sweat) collection from a skin surface. The film layer also includes an aperture that overlaps the adhesive layer aperture to direct collected fluid to the capillary channels of the microstructured surface, which have a hydrophilic surface. Optionally at least one indicator chemistry can be present in the channels to facilitate optical (e.g., visual) determination of total fluid volume and/or concentration of at least one analyte in the fluid as a function of time.

### BACKGROUND

There is growing interest in collecting biometric data using sensors worn on the body. A familiar example is activity trackers that detect motion using accelerometers worn on the wrist. An emerging field in wearable sensing is monitoring sweat utilizing skin-interfaced microfluidic devices. Example applications include determining sweat volume over time and measuring specific analytes in sweat via incorporation of biosensors in the fluidic device. Determining volume or detecting specific analytes can be achieved electrochemically, optically, or a combination of both. See, e.g., International Application Publication No. WO2019/023195 (Model et al.), U.S. Application Publication Nos. US2018/0303388 (Rao et al.), US2018/0042585 (Heikenfeld et al.), and US2019/0008448 (Begtrup et al.). There remains a need to provide articles capable of collecting fluid when worn on the body and providing information about the fluid.

### SUMMARY

In a first aspect, an article is provided. The article includes a) an adhesive layer; b) a film layer bonded to the adhesive layer having a microstructured surface including a plurality of capillary channels; and c) a cover layer disposed on the plurality of capillary channels of the film layer. More particularly, the adhesive layer has a first major surface and an opposing second major surface, and the adhesive layer defines a first aperture having at least one edge. The first major surface of the adhesive layer lacks spontaneous wicking when contacted with an aqueous fluid. The film layer has a first major surface and a second major surface, and the first major surface of the film layer is bonded to the first major surface of the adhesive layer. The film layer defines a second aperture disposed partially overlapping the first aperture. The second major surface of the film layer is the microstructured surface and each of the plurality of capillary channels is in fluid communication with the second aperture and extends toward a perimeter of the film layer. A surface of each of the capillary channels exhibits spontaneous wicking when contacted with an aqueous fluid.

It has been discovered that articles according to at least certain embodiments of this disclosure can provide a small skin-interfaced device that provides consistent, uniform collection and transportation of sweat.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1A is an exploded generalized schematic diagram of an exemplary article.

FIG. 1B is a generalized schematic diagram of an exemplary article according FIG. 1A.

FIG. 1C is a cross-section of the article of FIG. 1B.

FIG. 1D is a generalized schematic diagram of a portion of the film layer of an exemplary article.

FIG. 1E is a generalized schematic cross-section of another exemplary article according to FIG. 1A.

FIG. 2A is a cross-sectional schematic view of a film having a microstructured surface in which capillary channels are defined by sidewalls that converge together at a bottom of the capillary channel (e.g., V-shaped).

FIG. 2B is a cross-sectional schematic view of a film having a microstructured surface in which capillary channels are defined by sidewalls and a bottom wall between them.

FIG. 3 is a generalized schematic top view diagram of another exemplary article.

FIG. 4A is a generalized schematic top view diagram of a further exemplary article according to Example 2, prior to filling with fluid.

FIG. 4B is a generalized schematic top view diagram of the article of FIG. 4A after filling with fluid began.

FIG. 4C is a generalized schematic top view diagram of the article of FIG. 4A as filling with fluid continued.

FIG. 4D is a generalized schematic top view diagram of the article of FIG. 4A as filling with fluid further continued.

FIG. 5 is a generalized schematic top view diagram of an exemplary article including an opaque region and a viewing window region.

FIG. 6A is a generalized schematic top view diagram of an additional exemplary article.

FIG. 6B is an SEM image of a portion of capillary channels of an exemplary article having a discontinuous region on the surface.

While the above-identified figures set forth several embodiments of the disclosure other embodiments are also contemplated, as noted in the description. The figures are not necessarily drawn to scale. In all cases, this disclosure presents the invention by way of representation and not limitation.

### DETAILED DESCRIPTION OF ILLUSTRATIVE EMBODIMENTS

As used herein, the term “microreplication” means the production of a microstructured surface through a process where the structured surface features retain an individual feature fidelity during manufacture.

As used herein, the term “microstructure” encompasses both structures (i.e., features) that protrude above a major surface of a substrate, and structures that are recessed below a major surface of a substrate. Combinations of protruding and recessed features are contemplated. By a microstructure is further meant that the structure is a predetermined, molded structure (e.g., as obtained by molding a polymeric thermoplastic resin against a tooling surface that comprises the negative of the microstructure desired to be provided on a first major side of a substrate) with dimensions ranging from about 5 to about 3000 micrometers in at least two orthogonal directions. One of these orthogonal directions may often be normal to the plane of the substrate (e.g., along the z-axis,) thus this dimension can comprise, e.g., a protrusion height or a recess depth.

As used herein, the term “capillary channel” refers to a passageway through which a fluid flows without the assistance of external forces (e.g., pressure, gravity, vacuum, etc.).

As used herein, the term “glass transition temperature” (T_{g}), of a polymer refers to the transition of a polymer from a glassy state to a rubbery state and can be measured using Differential Scanning calorimetry (DSC), such as at a heating rate of 10° C. per minute in a nitrogen stream. When the T_{g} of a monomer is mentioned, it is the T_{g} of a homopolymer of that monomer. The homopolymer must be sufficiently high molecular weight such that the T_{g} reaches a limiting value, as it is generally appreciated that a T_{g} of a homopolymer will increase with increasing molecular weight to a limiting value. The homopolymer is also understood to be substantially free of moisture, residual monomer, solvents, and other contaminants that may affect the T_{g}. A suitable DSC method and mode of analysis is as described in Matsumoto, A. et. al., J. Polym. Sci. A., Polym. Chem. 1993, 31, 2531-2539.

As used herein, the term “Vicat softening temperature” of a polymer refers to the determination of the softening point for a material that has no definite melting point. It is taken as the temperature at which the specimen is penetrated to a depth of 1 mm by a flat-ended needle under a specific load.

As used herein, the term “hydrophilic” refers to a surface that is wet by aqueous solutions and does not express whether or not the material absorbs aqueous solutions. By “wet” it is meant that the surface exhibits spontaneous wicking when contacted with an aqueous fluid. An aqueous fluid comprises 50% or more by volume water. In some embodiments, a hydrophilic surface exhibits an advancing (maximum) water contact angle of less than 90°, preferably 45° or less.

As used herein, the term “hydrophobic” refers to a surface that lacks spontaneous wicking when contacted with an aqueous fluid. In some embodiments, a hydrophobic surface exhibits an advancing water contact angle of 70° or greater, preferably 90° or greater.

As used herein, “curing” means the hardening or partial hardening of a composition by any mechanism, e.g., by heat, light, radiation, e-beam, microwave, chemical reaction, or combinations thereof. As used herein, the term “hardenable” refers to a material that can be cured or solidified, e.g., by heating to remove solvent, heating to cause polymerization, chemical crosslinking, radiation-induced polymerization or crosslinking, or the like. As used herein, “cured” refers to a material or composition that has been hardened or partially hardened (e.g., polymerized or crosslinked) by curing.

As used herein, a polymeric “film” is a polymer material in the form of a generally flat sheet that is sufficiently flexible and strong to be processed in a roll-to-roll fashion. By roll-to-roll, what is meant is a process where material is wound onto or unwound from a support, as well as further processed in some way. Examples of further processes include coating, slitting, blanking, and exposing to radiation, or the like. Polymeric films can be manufactured in a variety of thicknesses, ranging in general from about 5 micrometers to 1000 micrometers.

As used herein, “thermoplastic” refers to a polymer that flows when heated sufficiently above its glass transition point and become solid when cooled. In contrast, “thermoset” refers to a polymer that permanently sets upon curing and does not flow upon subsequent heating. Thermoset polymers are typically crosslinked polymers.

As used herein, “transparent” refers to a material (e.g., a layer) that has at least 50% transmittance, 70% transmittance, or optionally greater than 90% transmittance over at least the 400 nanometer (nm) to 700 nm portion of the visible light spectrum.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

In this application, terms such as “a”, “an”, and “the” are not intended to refer to only a singular entity, but include the general class of which a specific example may be used for illustration. The terms “a”, “an”, and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise. The term “and/or” means one or all of the listed elements or a combination of any two or more of the listed elements.

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties). The term “substantially”, unless otherwise specifically defined, means to a high degree of approximation (e.g., within +/−10% for quantifiable properties) but again without requiring absolute precision or a perfect match. Terms such as same, equal, uniform, constant, strictly, and the like, are understood to be within the usual tolerances or measuring error applicable to the particular circumstance rather than requiring absolute precision or a perfect match.

In a first aspect, the present disclosure provides an article. The article comprises:
a) an adhesive layer having a first major surface and an opposing second major surface, wherein the adhesive layer defines a first aperture having at least one edge, wherein the first major surface of the adhesive layer lacks spontaneous wicking when contacted with an aqueous fluid;
b) a film layer having a first major surface and a second major surface, wherein the first major surface of the film layer is bonded to the first major surface of the adhesive layer, wherein the film layer defines a second aperture disposed partially overlapping the first aperture, wherein the second major surface of the film layer is a microstructured surface comprising a plurality of capillary channels each in fluid communication with the second aperture and extending toward a perimeter of the film layer, and wherein a surface of each of the capillary channels exhibits spontaneous wicking when contacted with an aqueous fluid; and
c) a cover layer disposed on the plurality of capillary channels of the film layer.

Articles according to at least certain embodiments of the present disclosure are capable of uniformly transporting liquids (e.g., water, sweat, or other aqueous solutions) into the second aperture from the first aperture, spontaneously into the capillary channels, and spontaneously along the channels from the second aperture towards the perimeter of the film layer. This capability is often referred to as wicking. Two general factors that influence the ability of channels to spontaneously transport liquids are (i) the structure or topography of the surface (e.g., capillarity, shape of the cavity) and (ii) the nature of the surface (e.g., surface energy). To achieve the desired amount of fluid transport capability a designer may adjust the structure or topography of the film layer and/or adjust the surface energy of the capillary channel surfaces. In order to achieve wicking, a surface of the capillary channels must be capable of being “wet” by the liquid to be transported. Optionally, the susceptibility of a solid surface to be wet by a liquid is characterized by the contact angle that the liquid makes with the solid surface after being deposited on a horizontally disposed surface and allowed to stabilize thereon. This angle is sometimes referred to as the “static equilibrium contact angle,” and sometimes referred to herein merely as “advancing contact angle.” In some cases, a material is considered hydrophilic if it has an advancing contact angle of less than 90 degrees.

general, articles of the present disclosure include an adhesive layer containing a fluid collection aperture fluidically coupled to a film layer containing parallel capillary channels. The aperture in the adhesive provides a fluid collection zone of known cross-sectional area. A second aperture in the film layer is oriented generally perpendicular to the capillary channels, forming a feed channel that transports the fluid to individual microcapillaries (i.e., capillary channels) in a time-dependent sequence. Articles according to at least certain embodiments of the present disclosure provide a multilayered article that may be useful, for instance, for liquid sample acquisition. Suitable applications include, for example, medical wearables (e.g., sweat sensors).

Certain optical sweat sensors determine a total sweat volume as a function of time. This can be achieved by directing the sweat through a continuous microfluidic channel in a device adhered or mechanically attached to the skin. The volume of the channel is defined by its height, width, and length. During exercise or heat exposure, eccrine glands produce sweat at a known physiological rate. Accumulating sweat builds pressure in a hermetically sealed collection region in the device, forcing sweat to move in to the channel through a via and subsequently progress down the channel due to pressure-driven flow. Total sweat volume is determined by measuring the distance sweat has travelled down the channel length. Visualization of sweat can be aided by the addition of an indicator deposited in the channel In order to minimize the size of the device, channel configurations can be serpentine or spiral paths with the inlet end of the channel fluidically coupled to a sweat collection region and an exit end vented to atmosphere. An alternative configuration for volume determination that does not require a hermetic seal transports sweat via capillary action using absorbent or wicking materials such as paper or hydrophilic membranes. In such a configuration, a strip of paper or membrane is in fluid communication with a sweat collection region. Similar to the microfluidic channel, volume is determined by the distance sweat propagates through the wicking material via capillary action.

Another form of optical sweat sensor determines the concentration of an analyte in sweat. This can be achieved as a single measurement or in a sequential manner to monitor changes in concentration as a function of time. To determine analyte concentration as a function of time fluidically isolated analysis regions containing analytical reagents are required, which increases the complexity of such devices. An alternative to shunting liquid to isolated compartments is immobilization of detection reagents on a wicking membrane and allow sweat to flow through the membrane to a waste reservoir to ensure continuous flow over time.

Several challenges, however, exist in utilizing pressure flow or capillary action in wearable sweat sensing devices. For devices operating at constant sweat pressure, the flow rate is proportional to the cross-sectional area of the fluidic channel Small diameter channels have a lower flow rate than large channels. In addition, at constant pressure the flow rate decreases as a function of distance in the channel One solution is to produce large channels of shorter length, however the liquid in large channels can be dislodged to undesired locations by the acceleration and deceleration of the device caused by motion during use (e.g., G-force). For example, on a forearm of a person exercising, G-forces of 2 G to 15 G, such as 5 G to 10 G, may be imparted to the article.

For articles that will be used when a person is moving such that the article is subjected to G-forces of 5 G or greater, 6 G or greater, or 7 G or greater, it is preferable for the capillary channels to be configured to have a smaller cross-sectional area than articles that will be subjected to lower G-forces. For instance, a suitable cross-sectional area of each of the capillary channels to minimize loss of fluid out of the capillary channel due to G-forces may be 0.025 square millimeters (mm²) or greater, 0.030 mm² or greater, 0.035 mm² or greater, 0.040 mm² or greater, 0.045 mm² or greater, 0.050 mm² or greater, 0.055 mm² or greater, 0.060 mm² or greater, 0.065 mm² or greater, 0.070 mm² or greater, 0.075 mm² or greater, 0.080 mm² or greater, 0.085 mm² or greater, 0.090 mm² or greater, or 0.095 mm² or greater; and 0.400 mm² or less, 0.375 mm² or less, 0.350 mm² or less, 0.325 mm² or less, 0.300 mm² or less, 0.275 mm² or less, 0.250 mm² or less, 0.200 mm² or less, 0.175 mm² or less, 0.150 mm² or less, 0.125 mm² or less, 0.150 mm² or less, or 0.100 mm² or less. A length of the capillary channels may be fixed, and the cross-sectional area is modified by altering one or more of channel height or width. There may be a preferred orientation for a given cross-sectional area to optimize the aspect ratio for visualization of fluid in the capillary channels.

In contrast, for articles that will be used when a person is generally sedentary or moving such that the article is subjected to G-forces of less than 5 G, 4 G or less, 3 G or less, or 2 G or less, there tends to be greater flexibility in the cross-sectional area of each of the capillary channels with minimal risk of loss of fluid out of the capillary channel due to G-forces. For instance, a suitable cross-sectional area of each of the capillary channels may be 0.025 mm² or greater, 0.030 mm² or greater, 0.035 mm² or greater, 0.040 mm² or greater, 0.045 mm² or greater, 0.050 mm² or greater, 0.055 mm² or greater, 0.060 mm² or greater, 0.065 mm² or greater, 0.070 mm² or greater, 0.075 mm² or greater, 0.080 mm² or greater, 0.085 mm² or greater, 0.090 mm² or greater, 0.095 mm² or greater; and 0.500 mm² or less, 0.450 mm² or less, 0.450 mm² or less, 0.425 mm² or less, 10 0.400 mm² or less, 0.375 mm² or less, 0.350 mm² or less, 0.325 mm² or less, 0.300 mm² or less, 0.275 mm² or less, 0.250 mm² or less, 0.200 mm² or less, 0.175 mm² or less, 0.150 mm² or less, 0.125 mm² or less, 0.150 mm² or less, or 0.100 mm² or less.

The use of absorbent or wicking membranes can overcome some of the flow variability in microchannel systems, however, if sweat rate changes during use (e.g., during periods of rest), capillary action can continue to propagate liquid, leading to an erroneously high estimate of sweat volume. These challenges become more significant in devices for detecting analytes in sweat, where the requirements for controllably shunting sweat to discrete zones on the device generates complex flow dynamics and venting requirements.

Articles according to at least certain embodiments of the present disclosure provide a small device attachable to a subject's skin that provides consistent, uniform collection of sweat from the subject. Optionally, the article also delivers sweat to at least one analytical detection chamber. These advantages are achieved by providing a device using a combination of pressure- driven delivery of sweat to a series of parallel compartments filled by capillary action. Utilization of a short feed channel overcomes the problems associated with flow rate. As the feed channel (e.g., the second aperture) fills with sweat, aliquots of defined volume are sequentially pulled in to the capillary channels. Since each capillary is open on the terminal end there is no need to produce individual vent holes or lanes in the device during assembly. Total volume is determined by the number of capillary channels filled per unit time as defined by their length, width and height. Regarding any article of the present disclosure, the capillary channels can contain the same indicator chemistry, (e.g., a reagent to measure ion concentration change as a function of time) or can contain different reagents for the determination of multiple target analytes. Such a format provides simplified manufacturing using roll-based processes without requiring site-specific registration of reagent deposition.

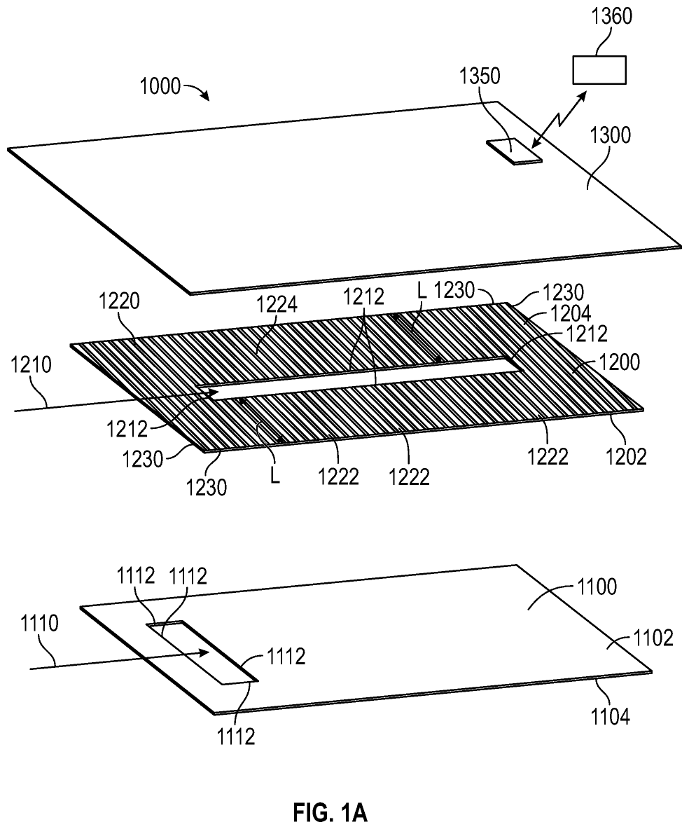

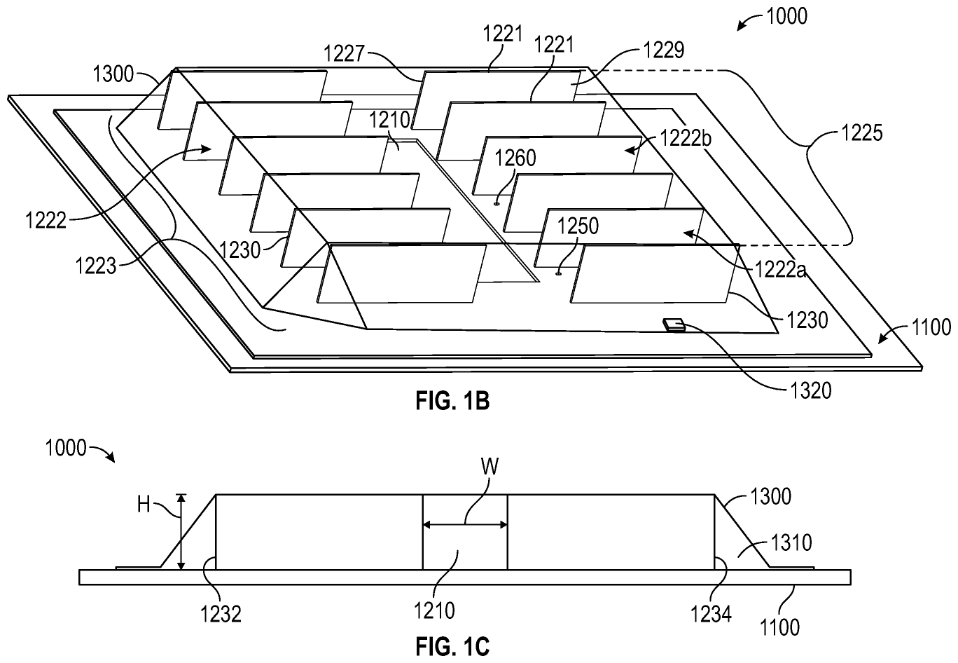

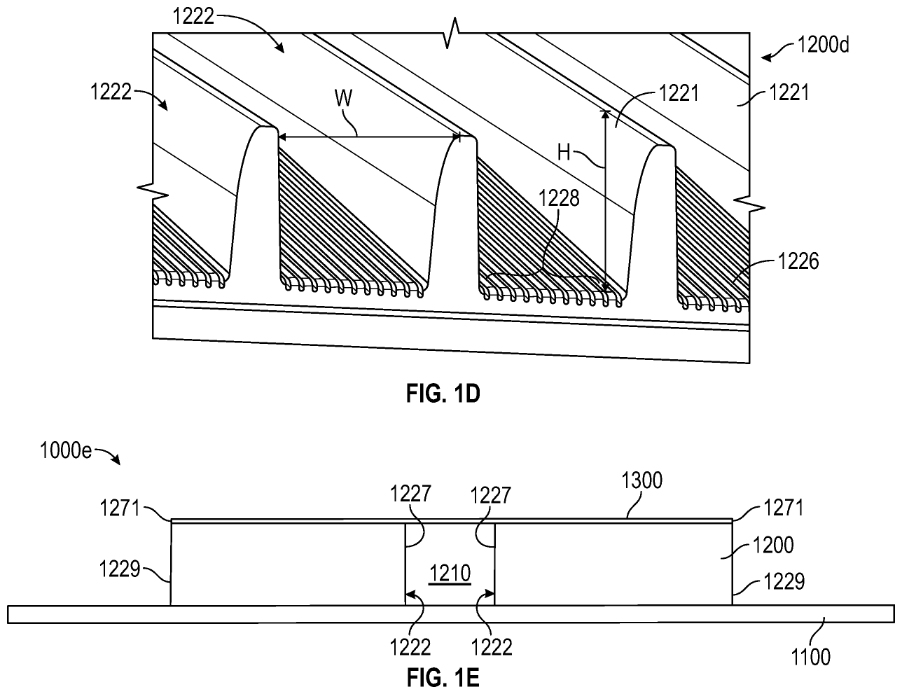

Exemplary articles according to at least certain embodiments of the present disclosure are shown in FIGS. 1A-1D. FIG. 1A is an exploded generalized schematic diagram of an exemplary article, FIG. 1B is a generalized schematic diagram of an exemplary article according to FIG. 1A, FIG. 1C is a cross-section of the article of FIG. 1B, FIG. 1D is a generalized schematic diagram of a portion of the film layer 1200 of an exemplary article, and FIG. 1E is a generalized schematic cross-section of another exemplary article according to FIG. 1A. An article 1000 comprises an adhesive layer 1100 having a first major surface 1102 and an opposing second major surface 1104, wherein the adhesive layer 1100 defines a first aperture 1110 having at least one edge 1112, which in this embodiment is a plurality of edges 1112. The first major surface 1104 of the adhesive layer 1100 lacks spontaneous wicking when contacted with an aqueous fluid (e.g., is hydrophobic). In certain embodiments, the at least one edge 1112 of the first aperture 1110 also lacks spontaneous wicking when contacted with an aqueous fluid.

Regarding any article described above, the first aperture optionally has an area of 0.25 square centimeters (cm²) or greater, 0.50 cm² or greater, 0.75 cm² or greater, 1.00 cm² or greater, 1.25 cm² or greater, 1.50 cm² or greater, 1.75 cm² or greater, 2.00 cm² or greater, 2.25 cm² or greater, or 2.50 cm² or greater; and 5.00 cm² or less, 4.75 cm² or less, 4.50 cm² or less, 4.25 cm² or less, 4.00 cm² or less, 3.75 cm² or less, 3.50 cm² or less, 3.25 cm² or less, 3.00 cm² or less, or 2.75 cm² or less. Stated another way, the first aperture may have an area ranging from 0.25 to 5.00 cm^{2,} inclusive. Advantageously, a low area assists in successful collection of fluid at a low flow rate and/or a low total fluid volume. The shape of the first aperture is not particularly limited, and optionally includes for instance, a circle, a square, a rectangle, a branched shape, or any combination of shapes.

Suitable materials for the adhesive layer include for instance a pressure-sensitive adhesive. The adhesive layer can be made by coating a film of an adhesive containing an adhesive polymer. Preferably, the adhesive comprises an adhesive polymer and a crosslinking agent. The term “adhesive polymer” used herein refers to a polymer which exhibits adhesion at ambient temperature (e.g., 20-25° Celsius). The adhesive polymer may be, for example, acrylic polymer, polyurethane, polyolefin, or polyester. In select embodiments, the adhesive layer comprises a double coated adhesive film. Some suitable commercially available double coated adhesive films are from 3M Company (St. Paul, MN) under the trade designations 3M Medical Silicone Tape 2477P and each of 3M Medical Tape 1509, 1510, 1513, 1522, 9874, and 9877.

The article 1000 further comprises a film layer 1200 having a first major surface 1202 and a second major surface 1204, wherein the first major surface 1202 of the film layer 1200 is bonded to the first major surface 1102 of the adhesive layer 1100. The film layer 1200 defines a second aperture 1210 disposed partially overlapping the first aperture 1110. By “partially overlapping” is meant that a portion of the area of the second aperture overlays a portion or all of the area of the first aperture. The second major surface 1204 of the film layer 1200 is a microstructured surface 1220 comprising a plurality of capillary channels 1222 each in fluid communication with the second aperture 1210 and extending toward a perimeter 1230 of the film layer 1200. In this embodiment, the capillary channels 1222 each extend in a direction normal to a longitudinal axis of the second aperture 1210. In alternate embodiments, however, one or more of the capillary channels may extend in one or more directions, for instance at least one angle other than 90° from the longitudinal axis of the second aperture, extending in a curved direction, or any combination thereof. A surface 1224 of each of the capillary channels 1222 exhibits spontaneous wicking when contacted with an aqueous fluid (e.g., is hydrophilic). In some embodiments, the second aperture 1210 has a plurality of edges 1212 that each lack spontaneous wicking when contacted with an aqueous fluid (e.g., are hydrophobic).

Regarding any article described above, the second aperture 1210 desirably has a width of 200 micrometers or greater, 300 micrometers or greater, 400 micrometers or greater, 500 micrometers or greater, 600 micrometers or greater, 700 micrometers or greater, or 800 micrometers or greater; and 1500 micrometers or less, 1400 micrometers or less, 1300 micrometers or less, 1200 micrometers or less, 1100 micrometers or less, 1000 micrometers or less, or 900 micrometers or less.

In many embodiments, the film layer is hermetically sealed to the adhesive layer, which minimizes leakage of fluid sample from between the adhesive layer and the film layer.

In the embodiments of FIGS. 1B and 1C, the article 1000 further comprises a cover layer 1300 disposed on the plurality of capillary channels 1222 of the film layer 1200. In these embodiments, the cover layer 1300 is attached to the first major surface 1102 of the adhesive layer 1100 adjacent to the perimeter 1230 of the film layer 1200. A height difference H between the film layer 1200 and the adhesive layer 1100 defines a gap 1310 between the perimeter 1230 of the film layer 1200 and the cover layer 1300. The cover layer 1300 defines at least one vent 1320 in fluid communication with the gap 1310. The vent 1320 allows gas (e.g., air) displaced from at least one of the second aperture 1210 or the capillary channels 1222 to exit the article 1000 during fluid collection and transport within the article 1000.

In the embodiment of FIG. 1E, in contrast, the article 1000e comprises a cover layer 1300 disposed on the plurality of capillary channels 1222 of the film layer 1200, for instance attached to the tops 1271 of at least some of the capillary channels 1222. The capillary channels 1222 each comprise an end 1227 in fluid communication with the second aperture 1210 and an opposing end 1229, which is open to the atmosphere, allowing venting of each capillary channel 1222.

Regarding any article described above, the cover layer preferably forms a hermetic seal to the tops of the capillary channels and is sufficiently large in area to cover the entirety of the second aperture of the film layer. A fluid feed channel is defined by the first major surface of the adhesive layer, the plurality of edges of the second aperture, and a major surface of the cover layer (taken together). In some embodiments, one or more of the edges of the second aperture, the major surface of the cover layer, or any combination thereof, lacks spontaneous wicking when contacted with an aqueous solution. In favored embodiments, each of the surfaces that form the fluid feed channel lacks spontaneous wicking when contacted with an aqueous solution.

In many embodiments of the article 1000, a first portion 1223 of the plurality of the capillary channels 1222 extend from the second aperture 1210 to a first edge 1232 at the perimeter 1230 of the film layer 1200 and a second portion 1225 of the plurality of the capillary channels 1222 extend from the second aperture 1210 to an opposing second edge 1234 at the perimeter 1230 of the film layer 1200.

Favorably, the article 1000 further comprises at least one reagent, preferably configured to react with a (e.g., fluid) sample and provide a response, e.g., selected from an electrochemical response, an optical response, a fluorescent response, a chemiluminescent response, a pH adjustment, or any combination thereof. The article 1000 may comprise a first reagent 1250 disposed in a first capillary channel 1222a. The article 1000 may also comprise a second reagent 1260 disposed in a second capillary channel 1222b. The first reagent and/or the second reagent is configured to react with a sample containing glucose, an electrolyte, a drug, a metabolite (e.g., a drug metabolite), or any combination thereof For instance, determining the concentration of ions in sweat during exercise or prolonged exposure to a hot environment can provide information on hydration status. In select embodiments, the first reagent and/or the second reagent includes an acid, a base, a buffer, or any combination thereof, to cause a change in the pH of the sample. Some suitable reagents include for instance and without limitation, fluorogenic or chromogenic indicators, electrochemical reagents, agglutination reagents, analyte specific binding agents, amplification agents such as enzymes and catalysts, photochromic agents, dielectric compositions, analyte specific reporters such as enzyme-linked antibody probes, DNA probes, RNA probes, fluorescent or phosphorescent beads, or any combination thereof.

In the embodiment shown in FIG. 1A, the article 1000 further comprises a sensor 1350. In any embodiment, the sensor 1350 is configured to communicate with a wireless network 1360. Accordingly, data from the sensor 1350 could be sent to a device (e.g., computer, mobile device, etc.) via the wireless network 1360 to provide a record of results after a subject wears the article 1000, such as at least one response from reaction of a reagent with the fluid collected from the subject.

Preferably, the capillary channels 1222 are uniform and regular along substantially each channel length L. In many embodiments, at least one of the capillary channels 1222 is comprised of sidewalls 1221 (e.g., two sidewalls) that are configured to define the capillary channel 1222, and the sidewalls 1221 extend continuously from one end 1227 of that channel 1222 to an opposing end 1229 of that channel 1222. Referring to FIG. 1D, a diagram is provided of a portion of the film layer 1200 of an exemplary article. In the diagram, some visible features of the film layer 1200 include several capillary channels 1222. The capillary channels are each defined by (e.g., two) sidewalls 1221 and a bottom wall 1226. In this embodiment, the bottom wall 1226 is formed of a plurality of smaller, secondary channels 1228.

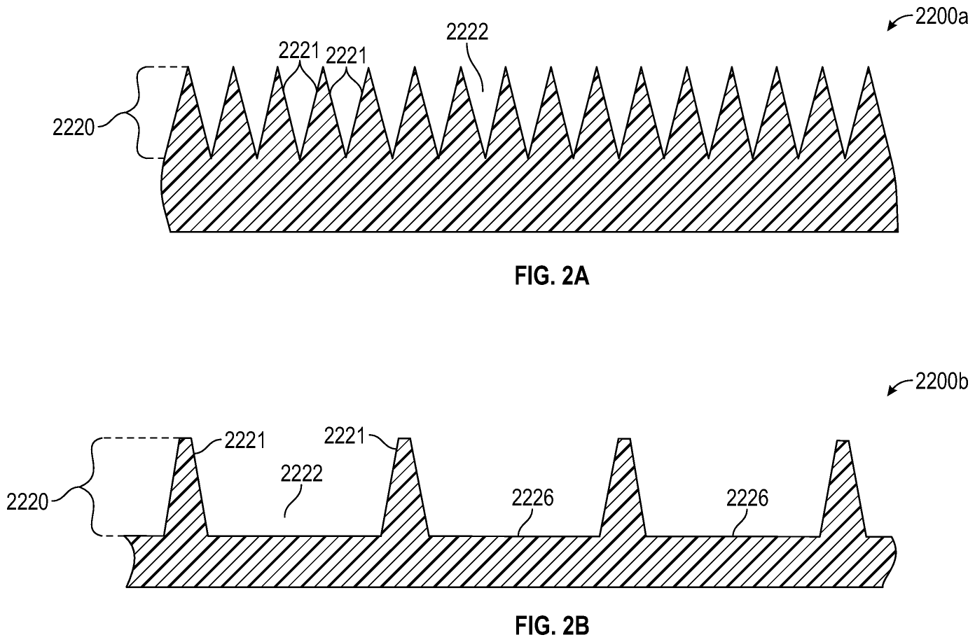

Referring to FIG. 2A, a cross-sectional schematic view is provided of a film 2200a having a microstructured surface 2220 in which the capillary channels 2222 are defined by sidewalls 2221 that converge together at a bottom of each capillary channel 2222 (e.g., the channels are V-shaped). FIG. 2B is a cross-sectional schematic view of another film 2200b having a microstructured surface 2220 in which the capillary channels 2222 are defined by sidewalls 2221 and a bottom wall 2226 between the sidewalls 2221. In preferred embodiments, a first side wall and a second side wall of a capillary channel are configured to each intersect with the bottom wall at an angle of less than 90 degrees, to assist in capillary movement of a sample entering the channel Additional suitable designs of capillary channels of microstructured surfaces useful for any article of the present disclosure are described in detail, e.g., in U.S. Pat. No. 7,223,364 (Johnston et al.), for instance as shown in FIGS. 1a-1h, 9, and 10a-10c.

Regarding any article described above, the capillary channels (1222, 2222) desirably each have a height H of 100 micrometers or greater, 200 micrometers or greater, 300 micrometers or greater, 400 micrometers or greater, 500 micrometers or greater, 600 micrometers or greater, or 700 micrometers or greater; and 3000 micrometers or less, 2500 micrometers or less, 2000 micrometers or less, 1500 micrometers or less, 1200 micrometers or less, 1000 micrometers or less, 900 micrometers or less, or 800 micrometers or less. It has been found that capillary channels having a larger height tend to provide more visually noticeable optical results than capillary channels having a smaller height.

Regarding any article described above, the capillary channels (1222, 2222) desirably each have a width W of 20 micrometers or greater, 30 micrometers or greater, 40 micrometers or greater, 50 micrometers or greater, 60 micrometers or greater, 80 micrometers or greater, 100 micrometers or greater, 125 micrometers or greater, 150 micrometers or greater, 175 micrometers or greater, 200 micrometers or greater, 250 micrometers or greater, 300 micrometers or greater, 350 micrometers or greater, 400 micrometers or greater, 450 micrometers or greater, or 500 micrometers or greater; and 1500 micrometers or less, 1400 micrometers or less, 1300 micrometers or less, 1200 micrometers or less, 1100 micrometers or less, 1000 micrometers or less, 900 micrometers or less, 800 micrometers or less, 700 micrometers or less, or 600 micrometers or less. It has been found that capillary channels having a smaller width are less likely to experience fluid moving irregularly within the channels (e.g., sloshing) than capillary channels having a larger width, when the article is subjected to motion (e.g., subject movement, which may subject the article to measurable G-forces), as discussed above. In select embodiments, when a larger height is employed a smaller width may be selected, and vice versa, for instance to provide a suitable total cross-sectional area (e.g., as described above) and/or volume (e.g., as described below), for each capillary channel

Regarding any article described above, the capillary channels (1222, 2222) desirably each have a volume of 0.25 microliters per centimeter (of length of the channel) or greater, 0.50 microliters per centimeter or greater, 0.75 microliters per centimeter or greater, 1.00 microliters per centimeter or greater, 1.25 microliters per centimeter or greater, 1.50 microliters per centimeter or greater, 1.75 microliters per centimeter or greater, 2.00 microliters per centimeter or greater, 2.25 microliters per centimeter or greater, 2.50 microliters per centimeter or greater, 2.75 microliters per centimeter or greater, 3.00 microliters per centimeter or greater, 3.25 microliters per centimeter or greater, 3.50 microliters per centimeter or greater, 3.75 microliters per centimeter or greater, or 4.00 microliters per centimeter or greater; and 10.00 microliters per centimeter or less, 9.50 microliters per centimeter or less, 9.00 microliters per centimeter or less, 8.50 microliters per centimeter or less, 8.00 microliters per centimeter or less, 7.50 microliters per centimeter or less, 7.00 microliters per centimeter or less, 6.50 microliters per centimeter or less, 6.00 microliters per centimeter or less, 5.75 microliters per centimeter or less, 5.50 microliters per centimeter or less, 5.25 microliters per centimeter or less, 5.00 microliters per centimeter or less, 4.75 microliters per centimeter or less, or 4.50 microliters per centimeter or less. Stated another way, the capillary channels may each have a volume of 0.25 microliters per centimeter to 10 microliters per centimeter. Advantageously, a low volume assists in successful transport of fluid through a capillary channel at a low flow rate and/or a low total fluid volume.

Similarly, the plurality of channels (1222, 2222) of any article described above optionally has a total volume of 0.1 milliliters (mL) or greater, 0.2 mL or greater, 0.3 mL or greater, 0.4 mL or greater, 0.5 mL or greater, 0.6 mL or greater, 0.7 mL or greater, 0.8 mL or greater, 0.9 mL or greater, 1.0 mL or greater, 1.1 mL or greater, 1.2 mL or greater, 1.3 mL or greater, 1.4 mL or greater, 1.5 mL or greater, 1.6 mL or greater, 1.7 mL or greater, 1.8 mL or greater, 1.9 mL or greater, 2.0 mL or greater, 2.1 mL or greater, 2.2 mL or greater, 2.3 mL or greater, or 2.4 mL or greater; and 5.0 mL or less, 4.8 mL or less, 4.6 mL or less, 4.4 mL or less, 4.2 mL or less, 4.0 mL or less, 3.8 mL or less, 3.6 mL or less, 3.4 mL or less, 3.2 mL or less, 3.0 mL or less, 2.9 mL or less, 2.8 mL or less, 2.7 mL or less, 2.6 mL or less, or 2.5 mL or less. Stated another way, the channels in totality may have a volume of 0.1 mL to 5.0 mL. Advantageously, a low total channel volume assists in successful filling of multiple capillary channels with fluid at a low flow rate and/or a low total fluid volume.

Regarding any article described above, the total volume provided by the plurality of capillary channels may be within 75%, 80%, 85%, 90%, or even within 95% of the total theoretical volume of the film layer defined by the perimeter length, width and height of the capillary features. Stated another way, an article favorably has less than 25%, 20% 15%, 10% or even less than 5% of the volume of the film layer occupied by the volume of the capillary channel walls. This relative area assists in providing an article that is desirably small and lacks wasted space while still providing uniform flow rate under constant pressure.

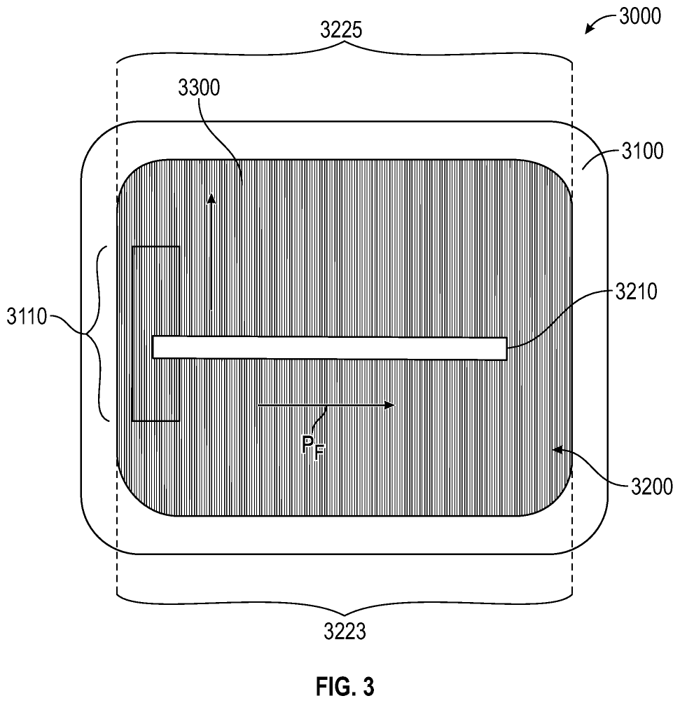

Referring to FIG. 3, a generalized schematic diagram is provided of another exemplary article 3000. The article 3000 comprises an adhesive layer 3100 to which a film layer 3200 is bonded, and a cover layer 3300 disposed on the film layer 3200. A first aperture 3110 of the adhesive layer 3100 is visible. A second aperture 3210 of the film layer 3200 is also provided. In an alternative design to the embodiment shown, the second aperture may extend all the way to an edge of the film layer to allow fluid to enter capillary channels down the full length of the film layer. A first portion 3223 of capillary channels and a second portion 3225 of capillary channels are shown. In use, once fluid fills the first aperture 3110, it will start to move through the second aperture 3210 and pressure PF will transfer aliquots of fluid into capillary channels of the first portion 3223 and the second portion 3225 in a direction towards a perimeter of the article 3000 at least because a hydrophilic surface of the capillary channels provides less resistance to the fluid than the hydrophobic surface of the adhesive layer 3100 below the second aperture 3210.

Regarding any article described above, suitable polymeric materials for the film layer include for instance and without limitation, a polyolefin (e.g., high density polyethylene (HDPE), medium density polyethylene (MDPE), or low density polyethylene (LDPE)), a polyester, a polyamide, a poly(vinyl chloride), a polyether ester, a polyimide, a polyesteramide, a polyacrylate, a polyvinylacetate, or a hydrolyzed derivative of polyvinylacetate. In certain embodiments, polyolefins are preferred because of their excellent physical properties, ease of processing (e.g., replicating the surface of a tool), and typically low cost. Also, polyolefins are generally tough, durable and hold their shape well, thus being easy to handle after article formation. In select embodiments, the film layer includes the polyester polyethylene terephthalate (PET). One suitable commercially available PET is a 5 mil (127 micrometer) thick PET sheet from Tekra (New Berlin, WI) under the trade designation “MELINEX 454”. A suitable commercially available LDPE is from The Dow Chemical Company (Midland, MI) under the trade designation “DOW 9551 LDPE”. Further, various additives may be included in the film layer, for example surface energy modifiers (such as surfactants and hydrophilic polymers), plasticizers, antioxidants, pigments, release agents, antistatic agents, and the like.

Regarding any article described above, the cover layer is optionally bonded to the tops of capillary channels of the film layer. One suitable method of bonding the cover layer to the capillary channels is using heat bonding (e.g., melt bonding). Further, a sensor may also be heat bonded to the capillary channels, the cover layer, or both. In embodiments using heat bonding, the film layer may be formed of a different polymeric material than the cover layer, including for instance and without limitation, a low density polyethylene, ethylene vinyl acetate, a polyurethane, copolymers of a polyester and a polyolefin, copolymers of a polyurethane and an aromatic poly(meth)acrylate, copolymers of a polycaprolactone and a polyurethane, or a combination thereof. One suitable commercially available polyurethane is from Lubrizol (Wickliffe, OH) under the trade designation “PEARLBOND 1160L”. A suitable commercially available ethylene vinyl acetate (EVA) is from The Dow Chemical Company under the trade designation “DUPONT ELVAX 3180”.

In some embodiments where the cover layer is bonded to the tops of capillary channels of the film layer, the film layer optionally has a Vicat softening temperature (T_{g}) of 100 degrees Celsius (° C.) or less, 95° C., 90° C., 85° C., 80° C., 75° C., or 70° C. or less; and 45° C. or more, 50° C., 55° C., 60° C., or 65° C. or more. The Vicat softening temperature of the film layer is often at least 10% lower than the Vicat softening temperature of the cover layer, 15% lower, 20% lower, 25% lower, 30% lower, 35% lower, or at least 40% lower than the Vicat softening temperature of the cover layer. In certain embodiments, the cover layer has a Vicat softening temperature (T_{g}) of 150 degrees Celsius (° C.) or less, 145° C., 140° C., 130° C., 120° C., 115° C., or 110° C. or less; and 65° C. or more, 70° C., 75° C., 80° C., or 85° C. or more. Using a cover layer with a higher Vicat softening temperature than the film layer assists in heat bonding the two layers together while minimizing conformation of the cover layer to the shape of the capillary channels.

In some embodiments where the cover layer is bonded to the tops of capillary channels of the film layer, the cover layer optionally comprises an adhesive. For instance, the cover layer may have an adhesive coated on the major surface that is attached to the tops of capillary channels (e.g., a patterned adhesive or a generally complete coating of adhesive), or the cover layer may be a multilayer construction comprising an adhesive layer and a layer of at least one of the polymeric materials described above.

Hydrophilicity of the capillary channels according to any article described above can be achieved through one or more of material selection, additives included in the material, or surface treatment. In some embodiments, the capillary channels have a surface including a surfactant, a surface treatment, a hydrophilic polymer, or a combination thereof Suitable surfactants include for instance and without limitation, C8-C18 alkane sulfonates; C8-C18 secondary alkane sulfonates; alkylbenzene sulfonates; C8-C18 alkyl sulfates; alkylether sulfates; sodium laureth 4 sulfate; sodium laureth 8 sulfate; dioctylsulfosuccinate, sodium salt; lauroyl lacylate; stearoyl lactylate; or any combination thereof. One or more surfactants can be applied by conventional methods, such as by wiping a coating of the surfactant on the surface of the capillary channels and allowing the coating to dry. A suitable surface treatment includes a hydrophilic coating comprising plasma deposited silicon/oxygen materials and/or diamond-like glass (DLG) materials. Plasma deposition of each of silicon/oxygen materials and DLG material is described, for instance, in PCT Publication No. WO 2007/075665 (Somasiri et al.). Further, examples of suitable DLG materials are disclosed in U.S. Pat. No. 6,696,157 (David et al.), U.S. Pat. No. 6,881,538 (Haddad et al.), and U.S. Pat. No. 8,664,323 (Iyer et al.). Suitable hydrophilic polymers include for instance and without limitation, a polyester, a polyamide, a polyurethane, a poly(vinyl alcohol), a poly(alkylene glycol), a poly(alkylene oxide), a poly(vinyl pyrrolidone), a rubber elastomer, or any combination thereof.

In some embodiments, the adhesive layer, the film layer, the cover layer, or any combination thereof, are transparent to visible light (as defined above). Providing one or more transparent layers can be advantageous for certain applications in which a sample reaction can be optically detected through at least a portion of the article (e.g., the cover layer).

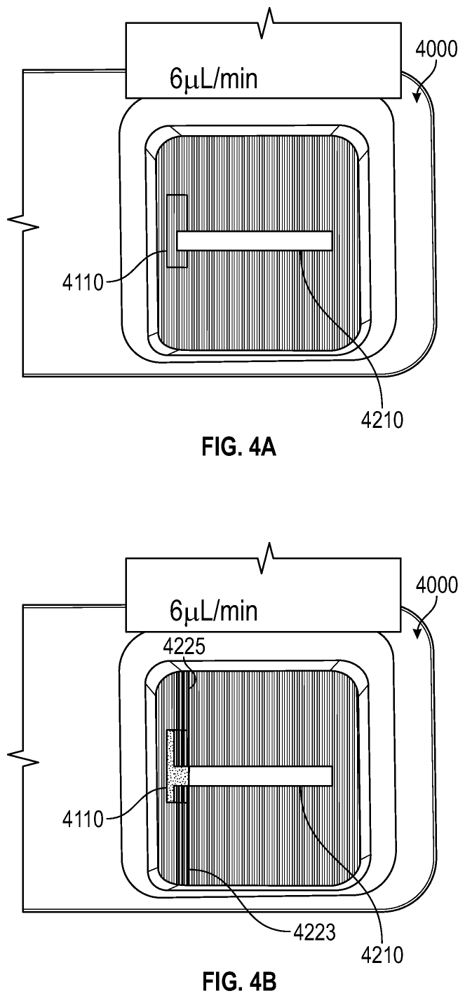

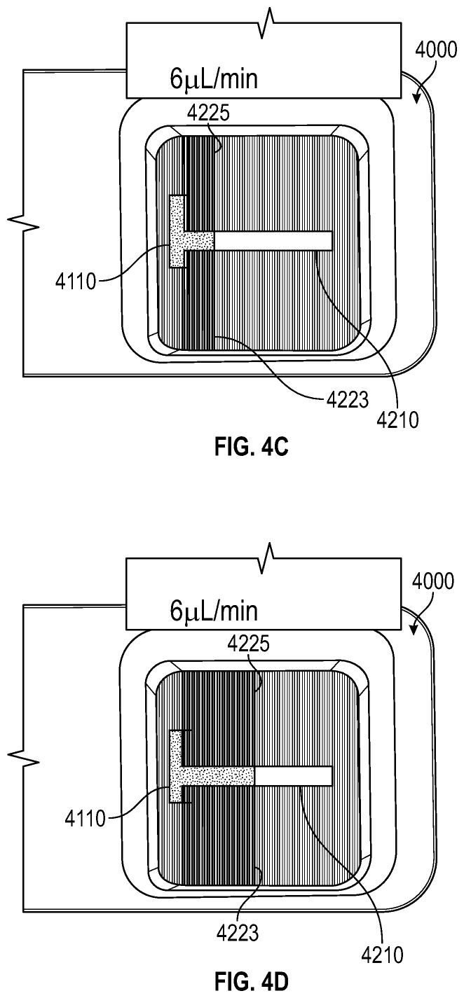

Referring to FIGS. 4A-4D, generalized schematic diagrams are shown of time lapse of an exemplary article 4000 in use. The use is described in detail below in Example 2. Briefly, FIG. 4A is a diagram of the article 4000 prior to filling with fluid (e.g., at a time of 0 seconds). FIG. 4B is a diagram of the article of FIG. 4A, 4 minutes and 55 seconds after filling with a colored fluid began, at a rate of 6 microliters per minute of the first aperture. It is apparent that the first aperture 4110 is full of fluid and that a very small number of the capillary channels of the first portion 4223 and of the second portion 4225 have been filled. FIG. 4C is a diagram of the article of FIG. 4A, 30 minutes and 55 seconds after filling with the colored fluid began. It is apparent that pressure of the fluid entering the first aperture at a rate of 6 microliters per minute of the first aperture is sufficient to continue to sequentially fill more capillary channels of the first portion 4223 and of the second portion 4225. FIG. 4D is a diagram of the article of FIG. 4A, 60 minutes and 55 seconds after filling with the colored fluid began, and even more capillary channels of the first portion 4223 and of the second portion 4225 have been sequentially filled by the fluid.

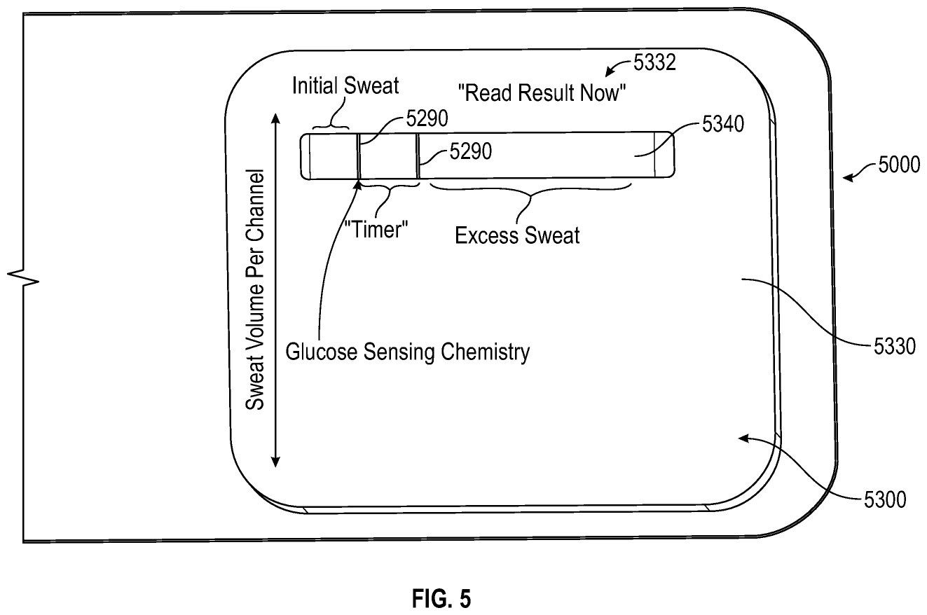

Regarding any article described above, the cover layer favorably further comprises an opaque region and a viewing window region. For example, referring to FIG. 5, an article 5000 is shown that has a cover layer 5300 that comprises an opaque region 5330 and a viewing window region 5340. The article 5000 of FIG. 5 further comprises indicia 5332 related to a volume of a sample, chemistry of a sample, or both. For instance, the article 5000 provides an optical response 5290 (i.e., a dark line in the viewing window region 5340) after reaction of a first reagent with at least a minimum amount of glucose in a subject's sweat. The article 5000 also provides an optical response 5290 based on reaction of a second reagent with a component of the subject's sweat to indicate that sufficient volume of the subject's sweat has been collected. Providing at least one optical response may be advantageous for any of the articles described above. It was unexpectedly discovered that little color was transferred from a first capillary channel to a subsequent capillary channel once the first capillary channel was filled, which allows successful use of multiple chemistries in different capillary channels.

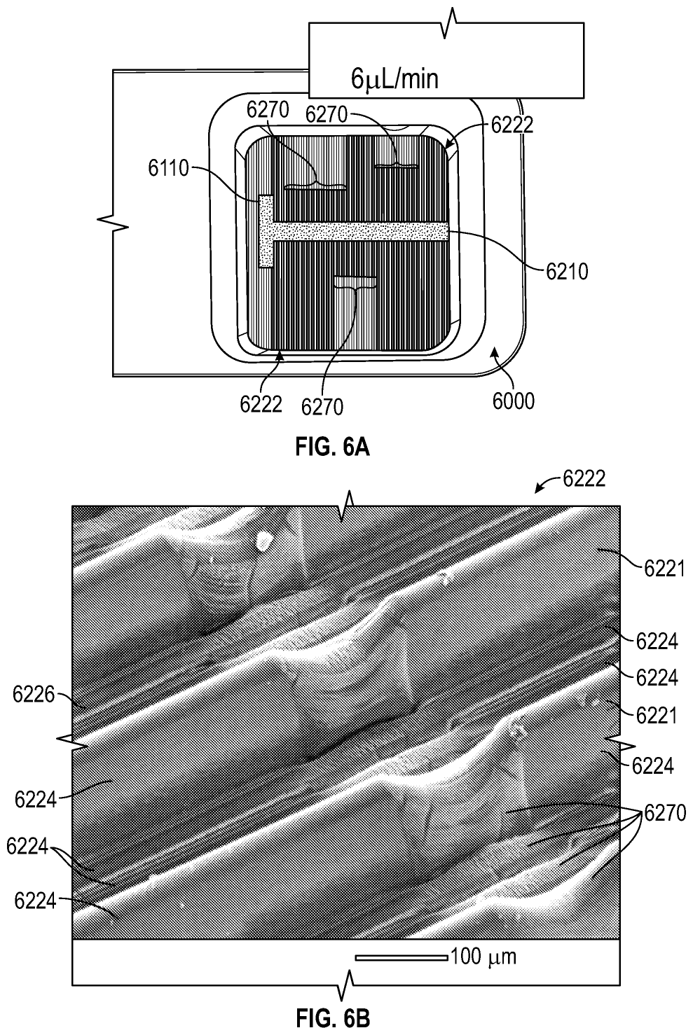

Referring to FIG. 6A, an article 6000 is shown that favorably further comprises a discontinuous region 6270 on the surface of at least one capillary channel 6222 that interrupts the surface that exhibits spontaneous wicking when contacted with an aqueous fluid (e.g., hydrophilic), wherein the discontinuous region 6270 comprises a surface that lacks spontaneous wicking when contacted with an aqueous fluid (e.g., hydrophobic). The article in the diagram comprises a discontinuous region 6270 in three different locations, each spanning a plurality of capillary channels 6222. The article has had a colored fluid transported through the first aperture 6110 and into the capillary channels 6222, which shows how the discontinuous hydrophobic region has stopped the flow of fluid through the capillary channels 6222 despite the remainder of the capillary channels 6222 beyond the discontinuous region having a hydrophilic surface. Occasionally the force of the fluid flow is sufficient to push past the discontinuous region 6270. Including such a discontinuous region can decrease the volume each capillary channel fills with fluid from the second aperture before fluid from the second aperture begins to fill an adjacent capillary channel This can be advantageous when it is expected that only a small amount of fluid will be collected from a subject. Referring to FIG. 6B, an SEM image is provided of a portion of capillary channels 6222 of an exemplary article having a discontinuous region 6270 on the surface of the capillary channels 6222. In the image, it can be seen that the (e.g., hydrophobic) discontinuous region 6270 is present on the sides on tops of each of the sidewalls 6221 and the bottom walls 6226 of the capillary channels 6222. The surfaces 6224 on either side of the discontinuous region 6270 exhibit spontaneous wicking when contacted with an aqueous fluid (e.g., are hydrophilic). A discontinuous region may be formed using laser ablation or mechanical cutting, for instance.

Regarding any article described above, a tie layer may be disposed between two adjacent layers (e.g., between the cover layer and the film layer). Some suitable tie layers are as described in U.S. Pat. No. 10,098,980 (Karls et al.), including a thermoplastic composition comprising a copolymer of at least one olefin monomer and at least one polar monomer and/or a block copolymer comprising alkyl methacrylate and alkyl acrylate blocks. The tie layer may be applied in a pattern.

### EXAMPLES

Objects and advantages of this disclosure are further illustrated by the following examples, but the particular materials and amounts thereof recited in these examples, as well as other conditions and details, should not be construed to unduly limit this disclosure. Unless otherwise noted or otherwise apparent from the context, all parts, percentages, ratios, etc. in the Examples and the rest of the specification are by weight. A materials table (below) lists materials used in the examples and their sources.

### Materials

| Material | Source |
|---|---|
| 4-Aminoantipyrine | Sigma-Aldrich Corporation, |
| | St. Louis, MO |
| 3-(N-Ethyl-methylanilino)-2- | Sigma-Aldrich Corporation |
| hydroxypropanesulfonic acid sodium salt | |
| Glucose oxidase/peroxidase solution | Sigma-Aldrich Corporation |
| Glucose standard solution (1 mg/mL) | Sigma-Aldrich Corporation |
| Phosphate buffered saline pH 7.4 (PBS) | Thermo Fisher Scientific, |
| | Waltham, MA |
| Artificial eccrine perspiration (product | Pickering Laboratories, |
| #1700-0022) | Mountain View, CA |
| Artificial apocrine sweat (product #1700- | Pickering Laboratories |
| 0556) | |

### Glucose Indicator Preparation

A solution was prepared containing 4-aminoantipyrine (1.5 mM) and 3-(N-ethyl-methylanilino)-2-hydroxypropanesulfonic acid sodium salt (1.5 mM) in PBS. A 1 mL aliquot of the solution was added to an Eppendorf tube and glucose oxidase/peroxidase solution (100 microliters) was added with mixing.

### Adhesive Layer Preparation

A rectangular section (6 cm long by 5 cm wide) of double coated double coated adhesive film was used as the adhesive layer (3M Double Coated Tape 9425HT, obtained from the 3M Corporation, St. Paul, MN). A 0.5 cm by 2 cm rectangular section was cut from the adhesive layer using a laser cutter (Muse Laser Cutter, obtained from Full Spectrum Laser, Las Vegas, NV) to form the first aperture. The first aperture was located at one end of the adhesive layer so that one long edge of the aperture was positioned 0.5 cm from an edge of the adhesive layer and the two narrow edges of the aperture were each positioned 1.5 cm from an edge of the adhesive layer.

### Film Layer Preparation

A polypropylene film (C700-35N, Dow Chemical Company, Midland, MI) with a microstructured surface containing a plurality of parallel capillary channels was prepared using an extrusion microreplication molding process as described in U.S. Pat. No. 10,378,813. The capillary channels of the microstructured surface consisted of primary walls having a height of about 600 micrometers spaced about 700 micrometers apart. The base of each channel had 10 secondary capillary channels formed by walls that had a height of about 35 micrometers, a top width of about 25 micrometers, and were spaced about 40 micrometers apart center to center. A hydrophilic silicon containing layer was applied to the microstructured surface using a Plasma-Therm 3032 batch plasma reactor (obtained from Plasma-Therm LLC, St. Petersburg, FL). The instrument was configured for reactive ion etching with a 26 inch (66.04 cm) lower powered electrode and central gas pumping. The chamber was pumped with a roots type blower (model EH1200 obtained from Edwards Engineering, Burgess Hill, UK) backed by a dry mechanical pump (model iQDP80 obtained from Edwards Engineering). The RF power was delivered by a 3 kW, 13.56 Mhz solid-state generator (RFPP model RF3OS obtained from Advanced Energy Industries, Fort Collins, CO). The system had a nominal base pressure of 5 mTorr. The flow rates of the gases were controlled by MKS flow controllers (obtained from MKS Instruments, Andover, MA). Samples of the film were fixed on the powered electrode of the plasma reactor. After pumping down to the base pressure, the gases tetramethylsilane (TMS) and oxygen (O₂) were introduced. The film was plasma treated in a 2 step process as follows: Step 1. TMS flow rate at 150 sccm (standard cubic centimeter per minute), oxygen flow rate at 500 sccm, and deposition time of 30 seconds; Step 2. oxygen at a flow rate of 500 sccm for 20 seconds. Following completion of the plasma treatment, the chamber was vented to the atmosphere and the plasma treated film was removed from the chamber.

A laser cutter (Muse Laser Cutter, obtained from Full Spectrum Laser, Las Vegas, NV) was used to create a rectangular section of plasma treated film (5 cm long by 4 cm wide) with the capillary channels oriented parallel to the width (short) dimension and extending to the edges of the section. A razor blade was used to cut a rectangular section of film approximately 1 mm wide from the center of the microchannel film, starting approximately 7 mm from one edge and extending to the opposite edge of the film. The removed section provided the second aperture which was oriented parallel to the length dimension and formed a narrow feed channel running perpendicular to the capillary channels.

### Example 1. Fluid Collection Article

A fluid collection article as shown in FIGS. 1A-1C was prepared using the adhesive layer and film layer prepared as described above. The non-microstructured surface of the film layer was adhesively attached to the adhesive layer. The layers were oriented so that the film layer was centered on the adhesive layer with the second aperture of the film layer partially overlapping (by about 2 mm) with the first aperture of the adhesive layer. This construction resulted in a 1 cm border of exposed adhesive along the outer edge of the assembly. A 6 cm by 5 cm section of TEGADERM Transparent film #16004 (obtained from the 3M Corporation) was used as the cover layer. The cover layer was positioned with the adhesive side facing the micro structured surface and was further edge aligned with the perimeter of the assembly. The cover layer was secured by adhesive attachment to the top surfaces of the channel walls and the exposed surface of the adhesive layer. The construction formed a continuous air gap around the perimeter, including the terminal ends of the capillary channels. A vent hole was created in the cover layer by a single puncture of the cover layer with a 20 gauge syringe needle. The vent hole was positioned over the air gap on the side of the assembly distal from the first aperture.

### Example 2. Delivery of a Liquid (Water) to a Fluid Collection Article

A fluid delivery apparatus was constructed containing a 1/16 inch (1.6 mm) channel bored through a 3/16 inch (4.8 mm) thick sheet of polycarbonate. The channel opening in the bottom surface was fluidically connected to a 1 mL syringe. A finished fluid control article of Example 1 was attached to the top surface of the apparatus using double sided tape. The article was oriented with the adhesive layer facing the apparatus surface and with the first aperture placed over the channel opening in the first surface. The apparatus was set-up to place the article in a flat, horizontal position. The syringe was filled with deionized water that contained a green food coloring agent. The syringe was placed in a syringe pump with the liquid delivered at a constant flow rate of 6 microliters per minute. The progression of liquid through the device was monitored using a video camera. The liquid was observed to initially fill the first aperture followed by liquid entry into the second aperture (feed channel). As the liquid filled the second aperture (feed channel), liquid sequentially entered capillary channels at the liquid front. Capillary channels were completely and continuously filled without any breaks or interruptions within a channel or between adjacent channels. The results for progression of liquid in the apparatus are presented in Table 1.

TABLE 1

| | |
|---|---|
| Time following the Start of Liquid | Total Number of Capillary |
| Filling | Channels Filled |

| | | |
|---|---|---|
| 1 | minute | 0 |
| 4 | minutes | 0 |
| 5 | minutes | 1 |
| 10 | minutes | 6 |
| 30 | minutes | 26 |
| 60 | minutes | 56 |

### Example 3. Delivery of a Liquid (Artificial Sweat) to Fluid Collection Articles (Horizontal Orientation of Capillary Channels)

The same procedure as described in Example 2 was followed with the exception that an artificial sweat solution (artificial eccrine perspiration #1700-0556 or artificial apocrine sweat #1700-0022 obtained from Pickering Laboratories, Inc) was used as the liquid. Both artificial sweat solutions filled the capillary channels of the article in the same complete, continuous and uninterrupted manner as described in Example 2. The results for progression of liquid in the apparatus using artificial apocrine sweat are presented in Table 2.

TABLE 2

| | |
|---|---|
| Time following the Start of Filling | Total Number of Capillary |
| with Artificial Apocrine Sweat | Channels Filled |

| | | |
|---|---|---|
| 1 | minute | 0 |
| 4 | minutes | 0 |
| 5 | minutes | 1 |
| 10 | minutes | 6 |
| 30 | minutes | 26 |
| 60 | minutes | 56 |

### Example 4. Delivery of a Liquid (Artificial Sweat) to Fluid Collection Articles (Vertical Orientation of Capillary Channels)

The same procedure as described in Example 3 was followed with the exception that the apparatus set-up was modified to orient the article in a vertical position. The artificial sweat solutions filled the capillary channels of the article in the same complete, continuous and uninterrupted manner as described in Example 3.

### Example 5. Colorimetric Indication of Glucose with a Fluid Collection Article

A 1 mL syringe equipped with a 25 gauge hypodermic needle was filled with glucose indicator solution (described above). A portion of the glucose indicator solution was added to fill a single capillary channel in a film layer. A separate 1 mL syringe equipped with a 25 gauge hypodermic needle was filled with an aqueous solution of green food coloring. A portion of the green indicator solution was added to fill a single capillary channel located downstream (about 0.5 cm) from the glucose indicator filled channel This channel was prepared to act as a liquid flow confirmation indicator. The film was placed under a stream of nitrogen to evaporate the solvents and form dry indicator coatings in the two channels. The film layer was then incorporated into a finished fluid collection article as described in Example 1. A second cover layer was applied over the TEGADERM cover layer. The second cover layer was an opaque, white vinyl tape (tape product #471, obtained from the 3M Company) that had a viewing window positioned over a portion of the capillary channels (including the indicator coated channels). A solution of glucose (standard solution diluted to a glucose concentration of 14 milligrams per deciliter) was introduced into the article at a flow rate of 6 microliters per minute according to the procedure described in Example 2. The obtained images were analyzed using ImagePro Plus image processing software (Media Cybernetics, Rockville, MD). The color density of the indicator coated channel was determined using the line profile tool. The color formation change was determined to be complete in approximately 30 minutes. Visual inspection of the glucose indicator coated channel showed a change from colorless to purple indicating a positive response for glucose in the injected fluid. Visual inspection of the channel coated with green coloring showed a change in color from light green to dark green indicating a positive response for liquid flow in the article.

### Example 6. Effect of Motion on Liquid Retention

Test articles for determining the effect of motion on liquid retention in the capillary channels were prepared in three layers according to the procedure of Example 1 with the exception that the adhesive layer did not have a first aperture and the film layer did not have a second aperture. The dimensions of the test articles were also varied. The overall capillary channel lengths were either 1 cm or 2 cm in length. In some test articles, films were used with channels having a smaller primary wall height (about 180 micrometers), primary wall spacing (about 180 micrometers), and only three secondary channels in each channel base. The microcapillary channels were then filled with water containing green food coloring. A 1 mL syringe with a 26 gauge needle was used to fill each channel with liquid. The channels were accessed by puncturing the cover sheet in the air gap section.

Prepared test articles were attached to a small aluminum plate using double sided tape with the surface of the coversheet exposed for visualization. The plate was secured to the forearm of a human subject using hook and loop attachment straps. The plate was attached to the forearm so that the long axis of the capillary channels was oriented parallel with gravity when the forearm was held in a position parallel to the floor. A data logging, three axis accelerometer (model PCE-VDL 16 L, obtained from PCE Instruments, Jupiter, FL) was mounted to the straps on the top of the forearm with the Z-axis detector oriented parallel to gravity. In some cases, the aluminum plate and accelerometer were held in the subject's hand (in the same orientation as described above).

The subject produced different motion conditions by pumping the arms with forward\backward motions while either walking, jogging, or sprinting. The maximum gravitational force (acceleration) was recorded for each condition. After ceasing motion, the test articles were visually analyzed to see if any liquid was released from the channels. The results are presented in Table 3.

TABLE 3

| | Wall | Wall | | | | Liquid |
|---|---|---|---|---|---|---|
| Channel | Spacing | Height | Test | | Acceler- | released |
| Length | (micro- | (micro- | Article | Motion | ation | from |
| (cm) | meters) | meters) | Location | Type | (G) | Channel |
| 2 | 700 | 600 | forearm | Jog | 3.2 | no |
| 2 | 700 | 600 | forearm | Run | 5.4 | yes |
| 1 | 700 | 600 | forearm | Jog | 3.2 | no |
| 1 | 700 | 600 | forearm | Run | 5.4 | no |
| 1 | 700 | 600 | forearm | Sprint | 7.0 | no |
| 1 | 700 | 600 | hand | Sprint | 12.0 | yes |
| 2 | 180 | 180 | forearm | Jog | 3.2 | no |
| 2 | 180 | 180 | forearm | Run | 4.7 | no |
| 2 | 180 | 180 | forearm | Sprint | 6.2 | no |
| 2 | 180 | 180 | hand | Sprint | 14.7 | no |

### Example 7. Fluid Collection Article with Discontinuous Regions

An article was prepared according to the procedure of Example 1 with the exception that prior to assembly of the article, sections of capillary channels in the film layer were laser cut with partial depth cuts to form discontinuous regions in the channels. Partial depth cuts were made perpendicular to the channel direction across multiple adjacent channels. Cuts were made at three different distances measured (0.5 cm, 1.0 cm, and 1.5 cm) from the edge of the second aperture (feed channel). Liquid (water with green food coloring) was injected into the article according to the procedure described in Example 2. The liquid was observed to continuously move through the capillary channels, but the liquid did stop within each channel at the location of a partial depth cut (illustrated in FIG. 6A).

All of the patents and patent applications mentioned above are hereby expressly incorporated by reference. The embodiments described above are illustrative of the present invention and other constructions are also possible. Accordingly, the present invention should not be deemed limited to the embodiments described in detail above and shown in the accompanying drawings, but instead only by a fair scope of the claims that follow along with their equivalents.

1. An article comprising:
a) an adhesive layer having a first major surface and an opposing second major surface, wherein the adhesive layer defines a first aperture having at least one edge, wherein the first major surface of the adhesive layer lacks spontaneous wicking when contacted with an aqueous fluid;
b) a film layer having a first major surface and a second major surface, wherein the first major surface of the film layer is bonded to the first major surface of the adhesive layer, wherein the film layer defines a second aperture disposed partially overlapping the first aperture, wherein the second major surface of the film layer is a microstructured surface comprising a plurality of capillary channels each in fluid communication with the second aperture and extending toward a perimeter of the film layer, and wherein a surface of each of the capillary channels exhibits spontaneous wicking when contacted with an aqueous fluid; and
c) a cover layer disposed on the plurality of capillary channels of the film layer.

2. The article of claim 1, wherein the cover layer is attached to the first major surface of the adhesive layer adjacent to the perimeter of the film layer, wherein a height difference between the film layer and the adhesive layer defines a gap between the perimeter of the film layer and the cover layer, and wherein the cover layer defines at least one vent in fluid communication with the gap.

3. The article of claim 1, wherein a first portion of the plurality of the capillary channels extend from the second aperture to a first edge at the perimeter of the film layer and a second portion of the plurality of the capillary channels extend from the second aperture to an opposing second edge at the perimeter of the film layer.

4. The article of claim 1, further comprising a first reagent disposed in a first capillary channel.

5. The article of claim 4, further comprising a second reagent disposed in a second capillary channel.

6. The article of claim 4, wherein the first reagent, the second reagent, or both is configured to react with a sample and provide a response selected from electrochemical, optical, fluorescent, chemiluminescent, pH adjustment, or a combination thereof.

7. The article of claim 6, wherein the first reagent, the second reagent, or both is configured to react with a sample containing glucose, an electrolyte, a drug, a metabolite, or a combination thereof.

8. The article of claim 1, wherein the at least one edge of the first aperture lacks spontaneous wicking when contacted with an aqueous fluid.

9. The article of claim 1, wherein the cover layer comprises an opaque region and a viewing window region.

10. The article of claim 9, wherein the opaque region comprises indicia related to volume of a sample, chemistry of a sample, or both.

11. The article of claim 1, wherein the cover layer further comprises a sensor.

12. The article of claim 1, wherein the capillary channels each have a height of 100 micrometers or greater, 200 micrometers or greater, 300 micrometers or greater, 400 micrometers or greater, 500 micrometers or greater, 600 micrometers or greater, or 700 micrometers or greater, and 3000 micrometers or less, 2500 micrometers or less, 2000 micrometers or less, 1500 micrometers or less, 1200 micrometers or less, 1000 micrometers or less, 900 micrometers or less, or 800 micrometers or less.

13. The article of claim 1, wherein the capillary channels each have a width of 20 micrometers or greater, 30 micrometers or greater, 40 micrometers or greater, 50 micrometers or greater, 60 micrometers or greater, 80 micrometers or greater, 100 micrometers or greater, 125 micrometers or greater, 150 micrometers or greater, 175 micrometers or greater, 200 micrometers or greater, 250 micrometers or greater, 300 micrometers or greater, 350 micrometers or greater, 400 micrometers or greater, 450 micrometers or greater, or 500 micrometers or greater; and 1500 micrometers or less, 1400 micrometers or less, 1300 micrometers or less, 1200 micrometers or less, 1100 micrometers or less, 1000 micrometers or less, 900 micrometers or less, 800 micrometers or less, 700 micrometers or less, or 600 micrometers or less.

14. The article of claim 1, wherein the capillary channels each has a volume of 0.25 microliters per centimeter to 10 microliters per centimeter.

15. The article of claim 1, wherein the plurality of capillary channels has a surface comprising a surfactant, a surface treatment, a hydrophilic polymer, or a combination thereof.

16. The article of claim 1, wherein the first aperture has an area ranging from 0.25 to 5.00 square centimeters.

17. The article of claim 1, wherein the plurality of capillary channels has a total volume of 0.1 milliliters to 5 milliliters.

18. The article of claim 1, wherein the film layer comprises a polyolefin, a polyester, a polyamide, a poly(vinyl chloride), a polyether ester, a polyimide, a polyesteramide, a polyacrylate, a polyvinylacetate, or a hydrolyzed derivative of polyvinylacetate.

19. The article of claim 1, further comprising a discontinuous region on the surface of at least one capillary channel that interrupts the surface that exhibits spontaneous wicking when contacted with an aqueous fluid, wherein the discontinuous region comprises a surface that lacks spontaneous wicking when contacted with an aqueous fluid.

20. The article of claim 1, wherein the first major surface of the adhesive layer, the second aperture, and a major surface of the cover layer together define a fluid feed channel and wherein the fluid feed channel lacks spontaneous wicking when contacted with an aqueous solution.
